(19) United States
(12) Patent Application Publication
(10) Pub. No.: US 2023/0410300 A1
(43) Pub. Date: Dec. 21, 2023

(54) IMAGE PROCESSING DEVICE, IMAGE PROCESSING METHOD, AND COMPUTER-READABLE RECORDING MEDIUM

(71) Applicant: OLYMPUS MEDICAL SYSTEMS CORP., Tokyo (JP)

(72) Inventors: Yuya TANAKA, Tokyo (JP); Hironao KAWANO, Tokyo (JP)

(73) Assignee: OLYMPUS MEDICAL SYSTEMS CORP., Tokyo (JP)

(21) Appl. No.: 18/242,179

(22) Filed: Sep. 5, 2023

Related U.S. Application Data
(63) Continuation of application No. PCT/JP2021/009679, filed on Mar. 10, 2021.

(51) Int. Cl. G06T 7/00; G06V 10/25; G06V 10/44

(52) U.S. Cl. CPC G06T 7/0012; G06V 10/25; G06V 10/44; G06T 2207/10068; G06T 2207/30096

(57) ABSTRACT

An image processing device includes: a processor comprising hardware, the processor being configured to calculate an evaluation value of a captured image that is obtained by capturing a subject, determine whether or not the evaluation value is included in a specific extraction range recorded in a memory, extract the captured image as an image of interest including a lesion when the processor has determined that the evaluation value is included in the specific extraction range, and update the specific extraction range based on the evaluation value.

### CROSS-REFERENCE TO RELATED APPLICATION

This application is a continuation of International Application No. PCT/JP2021/009679, filed on Mar. 10, 2021, the entire contents of which are incorporated herein by reference.

### BACKGROUND

### 1. Technical Field

The present disclosure relates to an image processing device, an image processing method, and a computer-readable recording medium.

### 2. Related Art

In a known endoscope system, a captured image of the interior of a subject is acquired by use of a swallowable capsule endoscope and a medical practitioner is thereby allowed to observe the captured image (see, for example, Japanese Unexamined Patent Application, Publication No. 2006-293237).

### SUMMARY

In some embodiments, an image processing device includes: a processor comprising hardware, the processor being configured to calculate an evaluation value of a captured image that is obtained by capturing a subject, determine whether or not the evaluation value is included in a specific extraction range recorded in a memory, extract the captured image as an image of interest including a lesion when the processor has determined that the evaluation value is included in the specific extraction range, and update the specific extraction range based on the evaluation value.

In some embodiments, an image processing method includes: calculating an evaluation value that is an evaluation of a degree of importance of a captured image that is obtained by capturing a subject; determining whether or not the evaluation value is included in a specific extraction range recorded in a memory; extracting the captured image as an image of interest including a lesion when it has been determined that the evaluation value is included in the specific extraction rang; and updating the specific extraction range based on the evaluation value.

In some embodiments, provided is a non-transitory computer-readable recording medium with an executable program stored thereon. The program causes a processor to execute: calculating an evaluation value that is an evaluation of a degree of importance of a captured image that is obtained by capturing a subject; determining whether or not the evaluation value is included in a specific extraction range recorded in a memory; extracting the captured image as an image of interest including a lesion when it has been determined that the evaluation value is included in the specific extraction range; and updating the specific extraction range based on the evaluation value.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an endoscope system according to a first embodiment;

FIG. 2 is a diagram illustrating a receiving device;

FIG. 3 is a diagram illustrating the receiving device;

FIG. 4 is a flowchart illustrating operation of the receiving device;

FIG. 5 is a diagram for explanation of Step S2;

FIG. 6 is a diagram illustrating a specific extraction range;

FIG. 7 is a flowchart illustrating operation of a receiving device according to a second embodiment;

FIG. 8 is a flowchart illustrating operation of a receiving device according to a third embodiment;

FIG. 9 is a flowchart illustrating operation of a receiving device according to a fourth embodiment;

FIG. 10 is a flowchart illustrating operation of a receiving device according to a fifth embodiment;

FIG. 11 is a diagram illustrating a specific example of a resetting operation;

FIG. 12 is a diagram illustrating the specific example of the resetting operation;

FIG. 13 is a diagram illustrating the specific example of the resetting operation;

FIG. 14 is a flowchart illustrating operation of a receiving device according to a sixth embodiment;

FIG. 15 is a diagram illustrating a specific extraction range;

FIG. 16 is a flowchart illustrating operation of a receiving device according to a seventh embodiment;

FIG. 17 is a flowchart illustrating operation of a receiving device according to an eighth embodiment;

FIG. 18 is a flowchart illustrating operation of a receiving device according to a ninth embodiment;

FIG. 19 is a flowchart illustrating operation of a receiving device according to a tenth embodiment;

FIG. 20 is a diagram illustrating a modified example of the first to fifth embodiments; and

FIG. 21 is a diagram illustrating a modified example of the sixth to tenth embodiments.

### DETAILED DESCRIPTION

Modes for implementing the disclosure (hereinafter referred to as embodiments) will be described hereinafter by reference to the drawings. The disclosure is not limited by the embodiments described hereinafter. Like portions will be assigned with like reference signs, throughout the drawings.

### First Embodiment

### Schematic Configuration of Endoscope System

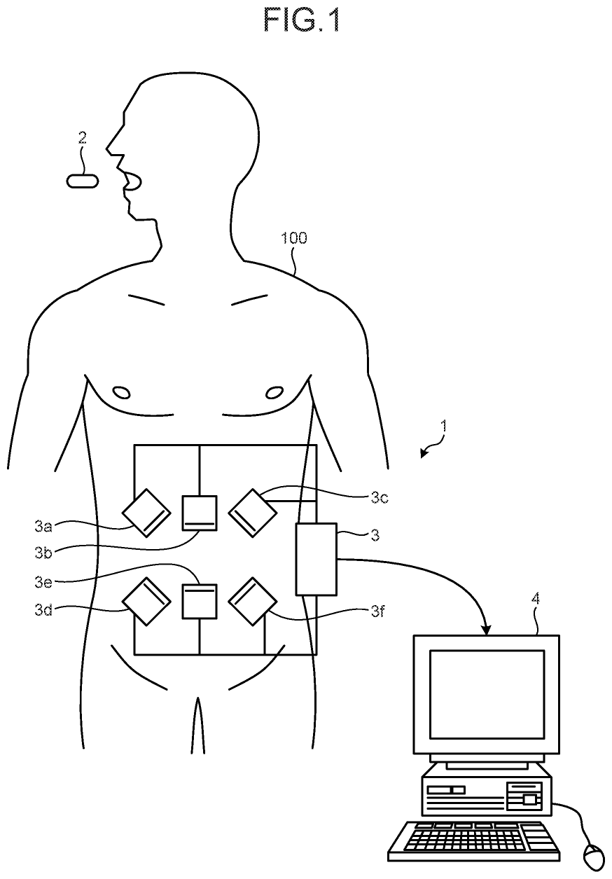

FIG. 1 is a diagram illustrating an endoscope system 1 according to a first embodiment.

The endoscope system 1 is a system for acquisition of a captured image of the interior of a subject 100 by use of a capsule endoscope 2 that is swallowable. The endoscope system 1 lets a user, such as a medical practitioner, observe the captured image.

This endoscope system 1 includes, as illustrated in FIG. 1, in addition to the capsule endoscope 2, a receiving device 3 and an image display device 4.

The capsule endoscope 2 is a capsule endoscope device formed in a size that enables the capsule endoscope device to be introduced into organs of the subject 100. The capsule endoscope 2 is introduced into the organs of the subject 100 by, for example, ingestion, and sequentially captures images while moving in the organs by, for example, vermicular movement. The capsule endoscope 2 sequentially transmits image data generated by capturing of the images.

The receiving device 3 corresponds to an image processing device. This receiving device 3 receives the image data from the capsule endoscope 2 inside the subject 100 via at least one of plural receiving antennas 3a to 3f each configured by use of, for example, a loop antenna or a dipole antenna. In this first embodiment, the receiving device 3 is used in a state of being carried by the subject 100, as illustrated in FIG. 1. The receiving device 3 is used in this way to reduce restrictions on activities of the subject 100 while the capsule endoscope 2 is inside the subject 100. That is, the receiving device 3 needs to continue receiving the image data transmitted to the receiving device 3 while the capsule endoscope 2 moves inside the subject 100 for a few hours to a few tens of hours, but keeping the subject 100 within a hospital over such a long period of time impairs user-friendliness brought about by use of the capsule endoscope 2.

Therefore, by downsizing the receiving device 3 to a size enabling the receiving device 3 to be portable in this first embodiment, freedom of activities of the subject 100 is obtained even while the capsule endoscope 2 is inside the subject 100 and burdens on the subject 100 are thus reduced.

The receiving antennas 3a to 3f may be arranged on a body surface of the subject 100 as illustrated in FIG. 1 or may be arranged in a jacket worn by the subject 100. The number of the receiving antennas 3a to 3f is not particularly limited to six and may be one or more.

A detailed configuration of the receiving device 3 will be described in a later section, “Configuration of Receiving Device”.

The image display device 4 is configured as a work station that acquires image data on the interior of the subject 100 from the receiving device 3 and displays images corresponding to the image data acquired.

Configuration of Receiving Device

The detailed configuration of the receiving device 3 will be described next.

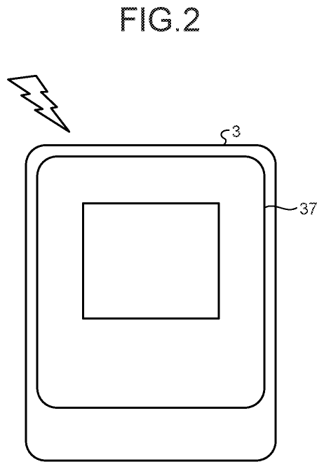

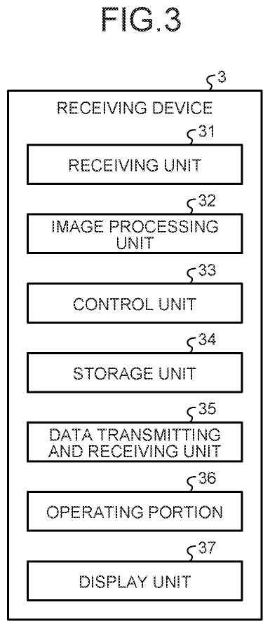

FIG. 2 and FIG. 3 are diagrams illustrating the receiving device 3.

The receiving device 3 includes, as illustrated in FIG. 2 or FIG. 3, a receiving unit 31 (FIG. 3), an image processing unit 32 (FIG. 3), a control unit 33 (FIG. 3), a storage unit 34 (FIG. 3), a data transmitting and receiving unit 35 (FIG. 3), an operating portion 36 (FIG. 3), and a display unit 37.

The receiving unit 31 receives the image data transmitted from the capsule endoscope 2 via at least one of the plural receiving antennas 3a to 3f.

The image processing unit 32 executes various types of image processing of the image data (digital signals) received by the receiving unit 31.

Examples of the image processing include optical black subtraction processing, white balance adjustment processing, digital gain processing, demosaicing processing, color matrix processing, gamma correction processing, and YC processing in which RGB signals are converted into luminance signals and color difference signals (Y, Cb/Cr signals).

The control unit 33 corresponds to a processor. The control unit 33 is configured by use of, for example, a central processing unit (CPU) or a field-programmable gate array (FPGA), and controls the overall operation of the receiving device 3, according to programs (including an image processing program) stored in the storage unit 34. Functions of the control unit 33 will be described in a later section, “Operation of Receiving Device”.

The storage unit 34 stores the programs (including the image processing program) executed by the control unit 33 and information needed in processing by the control unit 33. The storage unit 34 sequentially stores the image data that have been sequentially transmitted from the capsule endoscope 2 and subjected to the image processing by the image processing unit 32.

The data transmitting and receiving unit 35 is a communication interface and transmits and receives data to and from the image display device 4 by wire or wirelessly. For example, the data transmitting and receiving unit 35 transmits the image data stored in the storage unit 34, to the image display device 4.

The operating portion 36 is configured by use of an operating device, such as buttons or a touch panel, and receives user operations. The operating portion 36 outputs operation signals corresponding to the user operations, to the control unit 33.

The display unit 37 includes a display using, for example, liquid crystal or organic electroluminescence (EL), and displays images under control by the control unit 33.

In this first embodiment, the receiving device 3 has two display modes, a real time view mode and a playback view mode. These two display modes can be switched over to each other by user operations through the operating portion 36.

Specifically, in the real time view mode, images are sequentially displayed on the display unit 37, the images being based on image data that have been sequentially transmitted from the capsule endoscope 2 and subjected to the image processing by the image processing unit 32.

In the playback view mode, an image of interest extracted by the control unit 33 is displayed on the display unit 37.

Operation of Receiving Device

Operation of the receiving device 3 described above will be described next. The operation of the receiving device 3 corresponds to an image processing method.

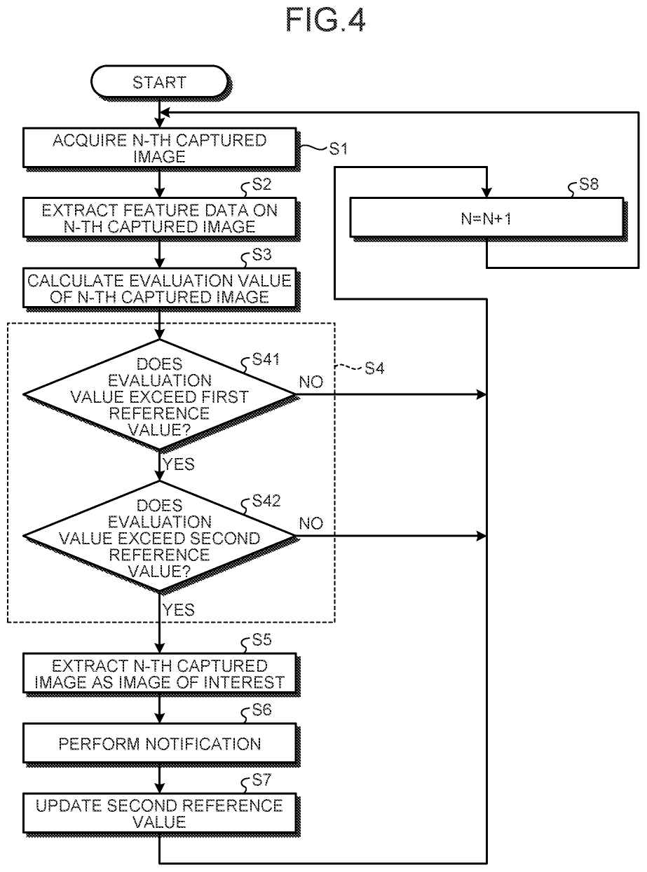

FIG. 4 is a flowchart illustrating the operation of the receiving device 3.

Firstly, the receiving unit 31 receives (acquires) data on an N-th image (hereinafter referred to as a captured image) transmitted from the capsule endoscope 2 (Step S1). The image processing unit 32 then executes image processing of the N-th captured image received by the receiving unit 31. The N-th captured image that has been subjected to the image processing is then stored in the storage unit 34.

After Step S1, the control unit 33 reads the N-th captured image stored in the storage unit 34 and extracts feature data on the N-th captured image (Step S2).

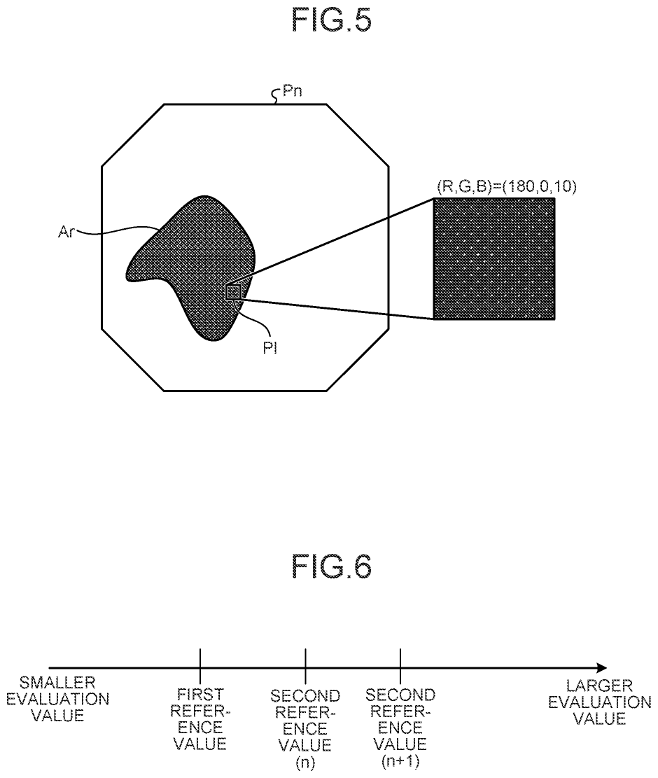

FIG. 5 is a diagram for explanation of Step S2. Specifically, FIG. 5 is a diagram illustrating an N-th captured image Pn. In FIG. 5, an area Ar that has been shaded represents, for example, a bleeding site captured in the N-th captured image Pn.

In this first embodiment, the control unit 33 calculates feature data for each of all of pixels of the N-th captured image Pn, at Step S2. Feature data herein mean feature data representing features of, for example, a bleeding site or a lesion captured in a captured image. Specifically, the control unit 33 calculates, as the feature data, for each of all of the pixels of the N-th captured image Pn, R/B resulting from division of R by B of its pixel values (R, G, B). For example, in a case where pixel values (R, G, B) of a specific pixel PI illustrated in FIG. 5 are (180, 0, 10), the control unit 33 calculates R/B=18 as feature data on that specific pixel PI.

After Step S2, the control unit 33 calculates an evaluation value of the N-th captured image Pn (Step S3).

Specifically, the control unit 33 compares R/B that is the feature data on each pixel with a specific reference value (for example, 10), at Step S3. The control unit 33 then calculates, as the evaluation value of the N-th captured image Pn, the number of pixels each having R/B exceeding the specific reference value, the pixels being among all of the pixels of the N-th captured image Pn.

After Step S3, the control unit 33 determines whether or not the evaluation value calculated at Step S3 is in a specific extraction range representing an image of interest (Step S4).

An image of interest herein means a captured image having a bleeding site or lesion captured therein, the captured image being needed to be used in diagnosis by a medical practitioner. An evaluation value is an index for extraction of a captured image as an image of interest.

FIG. 6 is a diagram illustrating the specific extraction range.

In this first embodiment, the specific extraction range is, as illustrated in FIG. 6, a range having a first reference value and a second reference value (n) larger than the first reference value, the range exceeding the first reference value and exceeding the second reference value (n). The initial value of the second reference value is a value that is at least equal to or larger than the first reference value.

Specifically, at Step S4, the control unit 33 determines whether or not the evaluation value exceeds the first reference value (Step S41).

In a case where the control unit 33 has determined that the evaluation value does not exceed the first reference value (Step S41: No), the receiving device 3 proceeds to Step S8.

On the contrary, in a case where the control unit 33 has determined that the evaluation value exceeds the first reference value (Step S41: Yes), the control unit 33 determines whether or not the evaluation value exceeds the second reference value (Step S42).

In a case where the control unit 33 has determined that the evaluation value does not exceed the second reference value (Step S42: No), the receiving device 3 proceeds to Step S8.

On the contrary, in a case where the control unit 33 has determined that the evaluation value exceeds the second reference value (Step S42: Yes), the control unit 33 extracts the N-th captured image Pn as an image of interest (Step S5).

Specifically, at Step S5, the control unit 33 associates information (hereinafter, referred to as interest information) with the N-th captured image Pn stored in the storage unit 34, the information indicating that the N-th captured image Pn is an image of interest.

After Step S5, the control unit 33 causes a notification of specific information to be made (Step S6).

Specifically, at Step S6, the control unit 33 causes the display unit 37 to display a message, such as “Please call a medical practitioner.” and causes sound to be output from a speaker (not illustrated in the drawings).

A method of making a notification of the specific information is not limited to the displaying of the message and outputting of the sound described above, and a method in which vibration is imparted to the subject 100 may be adopted.

After Step S6, the control unit 33 updates the specific extraction range (Step S7). Thereafter, the receiving device 3 proceeds to Step S8.

In this first embodiment, the control unit 33 updates the specific extraction range by changing the second reference value to a larger value, at Step S7. For example, as illustrated in FIG. 6, the control unit 33 changes the second reference value (n) that has been used thus far to a second reference value (n+1) larger than the second reference value (n).

From Step S8 onward, the receiving device 3 executes the processing of Steps S1 to S7 again for a captured image (N=N+1) subsequent to the N-th captured image Pn.

The above described first embodiment has the following effects.

In the receiving device 3 according to the first embodiment, the control unit 33 calculates an evaluation value of a captured image on the basis of the captured image. The control unit 33 then determines whether or not the evaluation value is in a specific extraction range representing an image of interest. Specifically, the control unit 33 determines that the evaluation value is in the specific extraction range in a case where the evaluation value exceeds a first reference value and the evaluation value exceeds a second reference value larger than the first reference value. The control unit 33 then extracts the captured image as an image of interest in a case where the control unit 33 has determined that the evaluation value is in the specific extraction range. In a case where the control unit 33 has determined that the evaluation value is in the specific extraction range, the control unit 33 updates the specific extraction range by changing the second reference value to a larger value.

It is now supposed, for example, that a captured image having an evaluation value exceeding a specific threshold is extracted as an image of interest. In this case, a captured image similar and temporally adjacent to the extracted captured image and not highly needed to be checked will be extracted as another image of interest.

In contrast, in this first embodiment, an image of interest is extracted by use of a specific extraction range and the specific extraction range is then updated as described above. Therefore, any captured image similar and temporally adjacent to the extracted image of interest and not highly needed to be checked will not be extracted as an image of interest and any representative captured image having, for example, a bleeding site captured therein is able to be extracted as an image of interest.

Therefore, the receiving device 3 according to the first embodiment enables extraction of an image of interest that is a captured image highly needed to be checked by a medical practitioner.

In particular, in this first embodiment, the receiving device 3 is configured as the image processing device. The receiving device 3 makes a notification of specific information in a case where a captured image has been extracted as an image of interest.

Therefore, performing a process of extracting a captured image as an image of interest in real time and making a notification of specific information in a case where the captured image has been extracted as the image of interest, at the receiving device 3, enable a medical practitioner to make a prompt decision on diagnostic principles for a subject.

Furthermore, the control unit 33 calculates feature data on a captured image on the basis of pixel values (R, G, B) of each pixel in the captured image.

Therefore, the feature data are able to be calculated by a simple process.

### Second Embodiment

A second embodiment will be described next.

In the following description, any component that is similar to that of the above described first embodiment will be assigned with the same reference sign, and detailed description thereof will be omitted or simplified.

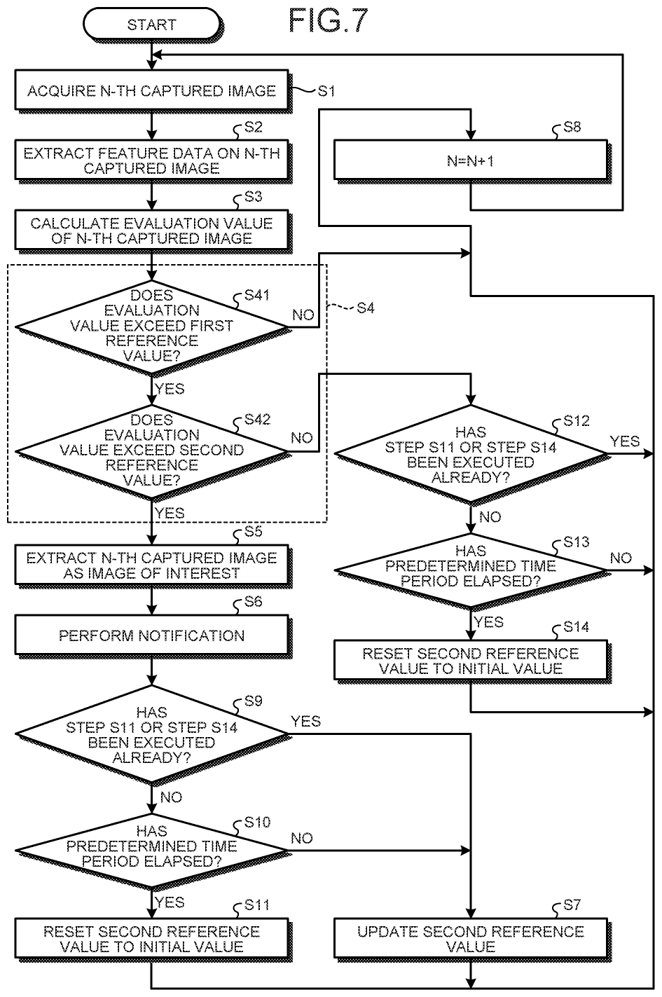

FIG. 7 is a flowchart illustrating operation of a receiving device 3 according to the second embodiment.

In this second embodiment, as illustrated in FIG. 7, the operation of the receiving device 3 is different from that of the first embodiment described above.

Specifically, the second embodiment is different from the first embodiment described above in that Steps S9 to S14 have been added in the second embodiment. Therefore, Steps S9 to S14 will be described mainly hereinafter.

Step S9 is executed after Step S6.

Specifically, at Step S9, a control unit 33 determines whether or not resetting of the second reference value to the initial value (Step S11 or Step S14 described later) has been executed already.

In a case where the control unit 33 has determined that the resetting of the second reference value to the initial value has been executed already (Step S9: Yes), the receiving device 3 proceeds to Step S7.

On the contrary, in a case where the control unit 33 has determined that the resetting of the second reference value to the initial value has not been executed yet (Step S9: No), the control unit 33 determines whether or not a predetermined time period has elapsed (Step S10). For example, at Step S10, the control unit 33 measures time from a time point at which data on a first image are received and determines whether or not the measured time has become equal to or longer the predetermined time period.

In a case where the control unit 33 has determined that the predetermined time period has not elapsed (Step S10: No), the receiving device 3 proceeds to Step S7.

On the contrary, in a case where the control unit 33 has determined that the predetermined time period has elapsed (Step S10: Yes), the control unit 33 reset the second reference value to the initial value (Step S11). Thereafter, the receiving device 3 proceeds to Step S8.

Step S12 is executed in a case where the control unit 33 has determined that the evaluation value does not exceed the second reference value (Step S42: No).

Specifically, at Step S12, the control unit 33 determines whether or not the resetting of the second reference value to the initial value (Step S11 or later described Step S14) has been executed already.

In a case where the control unit 33 has determined that the resetting of the second reference value to the initial value has been executed already (Step S12: Yes), the receiving device 3 proceeds to Step S8.

On the contrary, in a case where the control unit 33 has determined that the resetting of the second reference value to the initial value has not been executed yet (Step S12: No), the control unit 33 determines whether or not a predetermined time period has elapsed, similarly to Step S10 (Step S13).

In a case where the control unit 33 has determined that the predetermined time period has not elapsed (Step S13: No), the receiving device 3 proceeds to Step S8.

On the contrary, in a case where the control unit 33 has determined that the predetermined time period has elapsed (Step S13: Yes), the control unit 33 resets the second reference value to the initial value (Step S14). Thereafter, the receiving device 3 proceeds to Step S8.

The second embodiment described above has the following effects, in addition to effects similar to those of the first embodiment described above.

In a case where the capsule endoscope 2 captures an image of a red piece of clothing or a red wall, for example, before the subject 100 swallows the capsule endoscope 2, update of a specific extraction range that is not supposed to be updated will be executed at Step S7. In this case, a captured image having, for example, a bleeding site captured therein may fail to be extracted as an image of interest, the captured image being highly needed to be checked by a medical practitioner.

In the receiving device 3 according to the second embodiment, the second reference value is reset to the initial value in a case where the predetermined time period has elapsed.

Therefore, in the above mentioned case also, a captured image having, for example, a bleeding site captured therein is able to be extracted as an image of interest, the captured image being highly needed to be checked by a medical practitioner.

### Third Embodiment

A third embodiment will be described next.

In the following description, any component that is similar to that of the above described first embodiment will be assigned with the same reference sign, and detailed description thereof will be omitted or simplified.

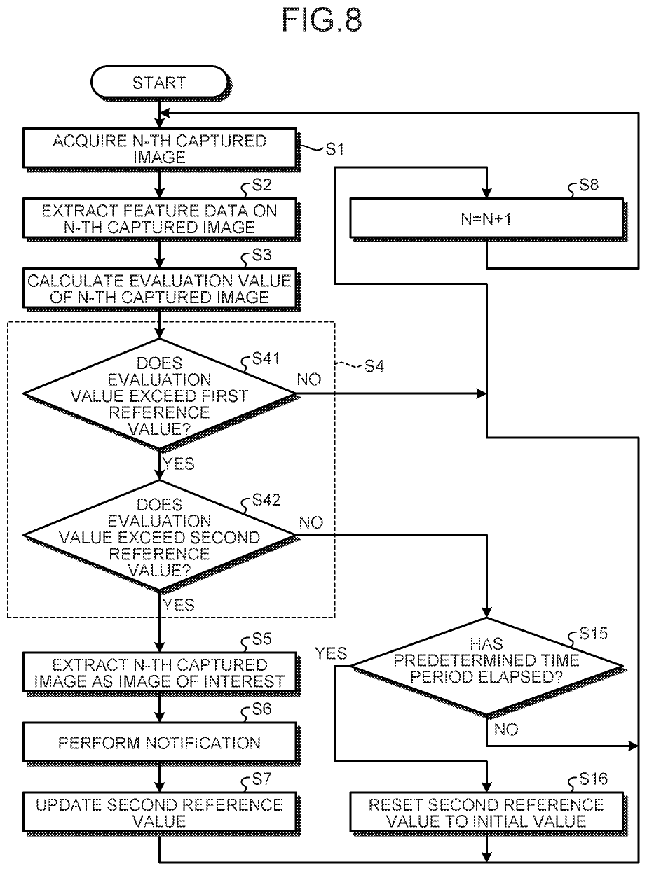

FIG. 8 is a flowchart illustrating operation of a receiving device 3 according to the third embodiment.

In this third embodiment, as illustrated in FIG. 8, the operation of the receiving device 3 is different from that of the first embodiment described above.

Specifically, the third embodiment is different from the first embodiment described above in that Steps S15 and S16 have been added in the third embodiment. Therefore, Steps S15 and S16 will be described mainly hereinafter.

Step S15 is executed in a case where it has been determined that the evaluation value does not exceed the second reference value (Step S42: No).

Specifically, at Step S15, a control unit 33 determines whether or not a predetermined time period has elapsed. For example, the control unit 33 measures a time period over which a state is maintained, the state being where the evaluation value exceeds the first reference value but does not exceed the second reference value, and the control unit 33 determines whether or not the time period measured has become equal to or longer than the predetermined time period.

In a case where the control unit 33 has determined that the predetermined time period has not elapsed (Step S15: No), the receiving device 3 proceeds to Step S8.

On the contrary, in a case where the control unit 33 has determined that the predetermined time period has elapsed (Step S15: Yes), the control unit 33 resets the second reference value to the initial value (Step S16). Thereafter, the receiving device 3 proceeds to Step S8.

The third embodiment described above has the following effects, in addition to effects similar to those of the first embodiment described above.

The capsule endoscope 2 may stagnate in the subject 100 or there may be plural bleeding sites and some of the bleeding sites may have less bleeding than a bleeding site that has been captured in a captured image extracted first as an image of interest. In such a case, a captured image having, for example, a bleeding site captured therein may fail to be extracted as an image of interest, the captured image being highly needed to be checked by a medical practitioner.

In the receiving device 3 according to the third embodiment, the second reference value is regularly reset to the initial value as the predetermined time period elapses.

Therefore, in the above mentioned case also, a captured image having, for example, a bleeding site captured therein is able to be extracted as an image of interest, the captured image being highly needed to be checked by a medical practitioner.

### Fourth Embodiment

A fourth embodiment will be described next.

In the following description, any component that is similar to that of the above described first embodiment will be assigned with the same reference sign, and detailed description thereof will be omitted or simplified.

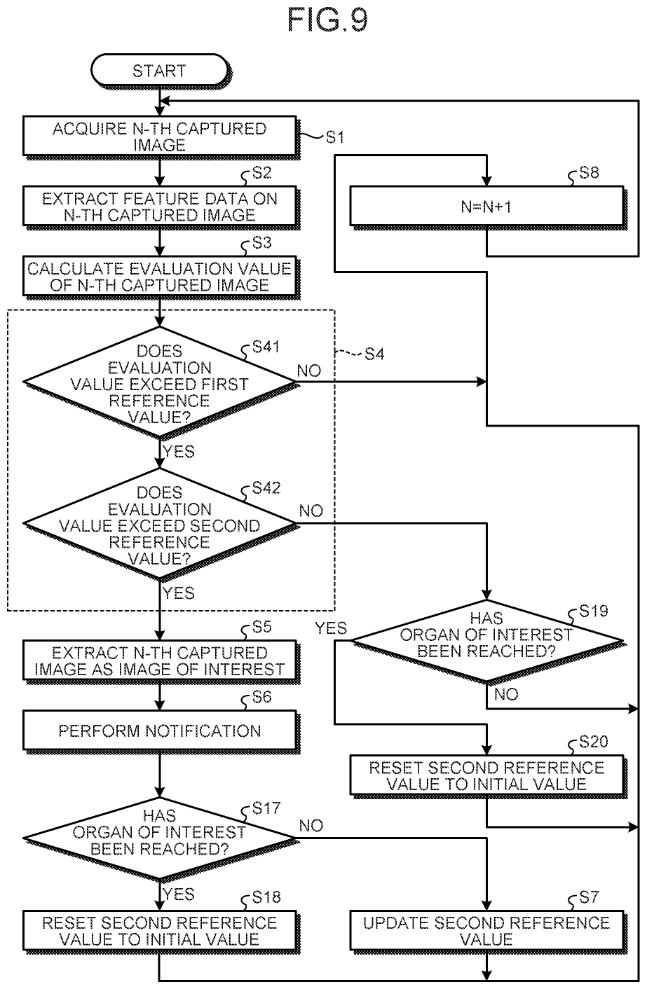

FIG. 9 is a flowchart illustrating operation of a receiving device 3 according to the fourth embodiment.

In this fourth embodiment, as illustrated in FIG. 9, the operation of the receiving device 3 is different from that of the first embodiment described above.

Specifically, the fourth embodiment is different from the first embodiment described above in that Steps S17 to S20 have been added in the fourth embodiment. Therefore, Steps S17 to S20 will be described mainly hereinafter.

Step S17 is executed after Step S6.

Specifically, at Step S17, a control unit 33 determines whether or not the capsule endoscope 2 has reached an organ of interest. This organ of interest means at least one specific organ that is an organ present in a path followed by the capsule endoscope 2 and that has been preset. For example, the control unit 33 determines whether or not the capsule endoscope 2 has reached the organ of interest, on the basis of a time period elapsed from a time point at which data on a first image are received, and/or a shape or color of a subject captured in an N-th captured image Pn.

In a case where the control unit 33 has determined that the capsule endoscope 2 has not reached the organ of interest (Step S17: No), the receiving device 3 proceeds to Step S7.

On the contrary, in a case where the control unit 33 has determined that the capsule endoscope 2 has reached the organ of interest (Step S17: Yes), the control unit 33 resets the second reference value to the initial value (Step S18). Thereafter, the receiving device 3 proceeds to Step S8.

Step S19 is executed in a case where the control unit 33 has determined that the evaluation value does not exceed the second reference value (Step S42: No).

Specifically, at Step S19, the control unit 33 determines whether or not the capsule endoscope 2 has reached the organ of interest, similarly to Step S17.

In a case where the control unit 33 has determined that the capsule endoscope 2 has not reached the organ of interest (Step S19: No), the receiving device 3 proceeds to Step S8.

On the contrary, in a case where the control unit 33 has determined that the capsule endoscope 2 has reached the organ of interest (Step S19: Yes), the control unit 33 resets the second reference value to the initial value (Step S20). Thereafter, the receiving device 3 proceeds to Step S8.

The fourth embodiment described above has the following effects, in addition to effects similar to those of the first embodiment described above.

It may sometimes be desired for a bleeding site to be checked for each of organs, such as a stomach and a small intestine, for example. However, in a case where Steps S1 to S8 have been executed repeatedly for captured images of the interior of the stomach, for example, the second reference value has been updated to a large value, and a captured image having a bleeding site captured therein may fail to be extracted as an image of interest, the bleeding site being in the small intestine reached after the stomach.

In the receiving device 3 according to the fourth embodiment, the second reference value is reset to the initial value every time the capsule endoscope 2 reaches an organ of interest.

Therefore, for each organ of interest, a captured image having, for example, a bleeding site captured therein is able to be extracted as an image of interest, the captured image being highly needed to be checked by a medical practitioner.

### Fifth Embodiment

A fifth embodiment will be described next.

In the following description, any component that is similar to that of the above described first embodiment will be assigned with the same reference sign, and detailed description thereof will be omitted or simplified.

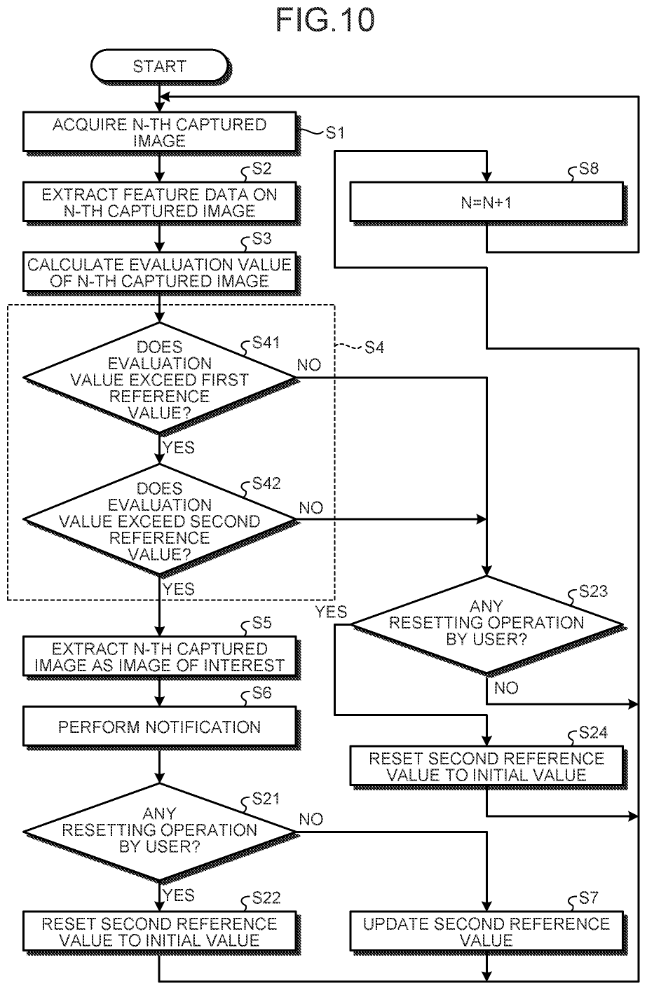

FIG. 10 is a flowchart illustrating operation of a receiving device 3 according to the fifth embodiment. In this fifth embodiment, as illustrated in FIG. 10, the operation of the receiving device 3 is different from that of the first embodiment described above.

Specifically, the fifth embodiment is different from the first embodiment described above in that Steps S21 to S24 have been added in the fifth embodiment. Therefore, Steps S21 to S24 will be described mainly hereinafter.

Step S21 is executed after Step S6.

Specifically, at Step S21, a control unit 33 determines whether or not a resetting operation (user operation) has been made through an operating portion 36 by a user.

In a case where the control unit 33 has determined that the resetting operation has not been made (Step S21: No), the receiving device 3 proceeds to Step S7.

On the contrary, in a case where the control unit 33 has determined that the resetting operation has been made (Step S21: Yes), the control unit 33 resets the second reference value to the initial value (Step S22). Thereafter, the receiving device 3 proceeds to Step S8.

Step S23 is executed in a case where the control unit 33 has determined that the evaluation value does not exceed the first reference value (Step S41: No), or in a case where the control unit 33 has determined that the evaluation value does not exceed the second reference value (Step S42: No).

Specifically, at Step S23, the control unit 33 determines whether or not a resetting operation (user operation) has been made through the operating portion 36 by a user.

In a case where the control unit 33 has determined that the resetting operation has not been made (Step S23: No), the receiving device 3 proceeds to Step S8.

On the contrary, in a case where the control unit 33 has determined that the resetting operation has been made (Step S23: Yes), the control unit 33 resets the second reference value to the initial value (Step S24). Thereafter, the receiving device 3 proceeds to Step S8.

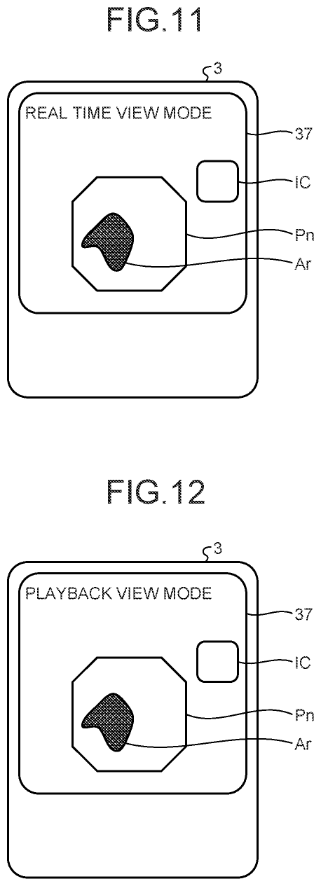

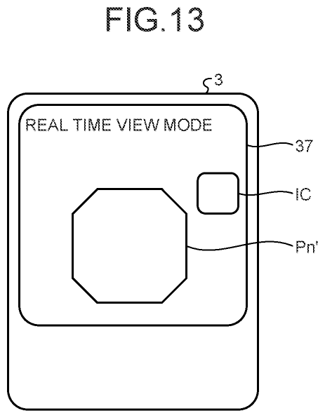

FIG. 11 to FIG. 13 are diagrams illustrating a specific example of the resetting operation. Specifically, FIG. 11 illustrates a state of the receiving device 3 upon execution of Step S6. FIG. 12 illustrates a state where a medical practitioner has switched a display mode of the receiving device 3 to a playback view mode after the execution of Step S6. FIG. 13 illustrates a state where a medical practitioner has switched the display mode of the receiving device 3 to a real time view mode after checking the state in FIG. 12. In FIG. 11 to FIG. 13, an icon IC displayed on a display unit 37 is an icon that is pressed by the resetting operation mentioned above.

In the real time view mode, captured images are sequentially displayed on the display unit 37, the captured images being based on image data that have been sequentially transmitted from the capsule endoscope 2 and subjected to image processing by an image processing unit 32. In a case where an N-th captured image Pn (FIG. 11) has been extracted as an image of interest at Step S6, the receiving device 3 makes a notification of specific information.

A medical practitioner checks the receiving device 3 according to the notification of the specific information from the receiving device 3. Specifically, the medical practitioner switches the display mode of the receiving device 3 to the playback view mode by a user operation through the operating portion 36. As illustrated in FIG. 12, the medical practitioner checks the N-th captured image Pn that is a captured image based on image data received in the past and that has been extracted as the image of interest.

After checking the N-th captured image Pn in the playback view mode, the medical practitioner switches the display mode of the receiving device 3 to the real time view mode by a user operation through the operating portion 36. The medical practitioner then checks whether there is any bleeding, as illustrated in FIG. 13, in a captured image Pn′ based on image data currently being received. In a case where the medical practitioner has been able to confirm a normal state without any bleeding, the medical practitioner presses the icon IC.

The fifth embodiment described above has the following effects, in addition to effects similar to those of the first embodiment described above.

In the receiving device 3 according to the fifth embodiment, the second reference value is reset to the initial value according to a resetting operation through the operating portion 36 by a user.

Therefore, a medical practitioner resets the second reference value to the initial value by a resetting operation in a case where the medical practitioner has confirmed a normal state without any bleeding in the real time view mode after checking the N-th captured image Pn in the playback view mode. A captured image having the next bleeding site captured therein, for example, is thereby able to be extracted as an image of interest.

### Sixth Embodiment

A sixth embodiment will be described next.

In the following description, any component that is similar to that of the above described first embodiment will be assigned with the same reference sign, and detailed description thereof will be omitted or simplified.

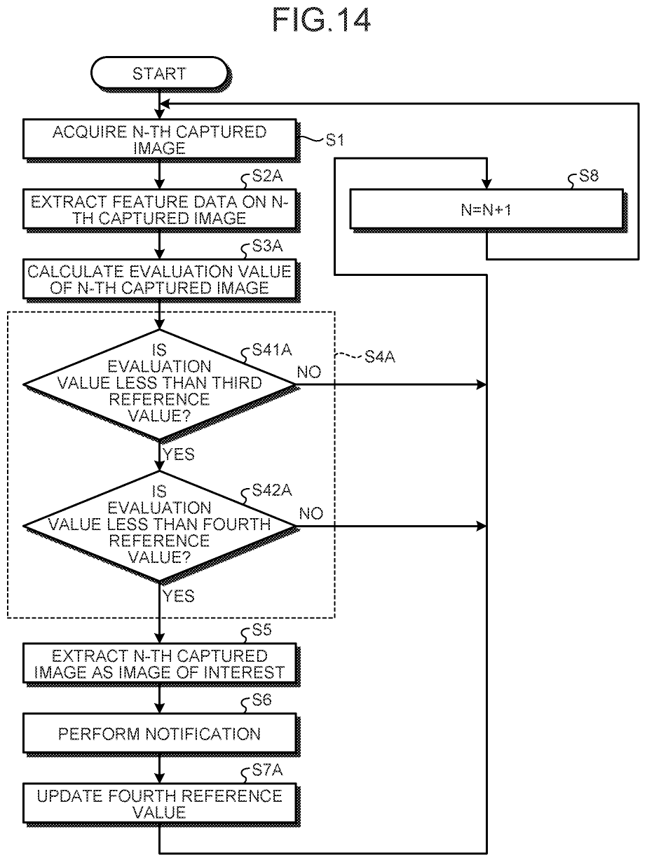

FIG. 14 is a flowchart illustrating operation of a receiving device 3 according to the sixth embodiment.

In this sixth embodiment, as illustrated in FIG. 14, the operation of the receiving device 3 is different from that of the first embodiment described above.

Specifically, the sixth embodiment is different from the first embodiment described above in that Steps S2A to S4A and S7A have been adopted in the sixth embodiment, instead of Steps S2 to S4 and S7. Therefore, Steps S2A to S4A and S7A will be described mainly hereinafter.

Step S2A

A control unit 33 calculates, as feature data, for each of all of pixels in an N-th captured image Pn, B/R resulting from division of B by R of the pixel values (R, G, B). For example, in a case where pixel values (R, G, B) of a specific pixel PI illustrated in FIG. 5 are (180, 0, 10), the control unit 33 calculates B/R=1/18 as feature data on that specific pixel PI.

Step S3A

The control unit 33 calculates an evaluation value of the N-th captured image Pn, the evaluation value being the smallest value of B/R of the featured data on the pixels.

Step S4A

The control unit 33 determines whether or not the evaluation value calculated at Step S3A is in a specific extraction range representing an image of interest.

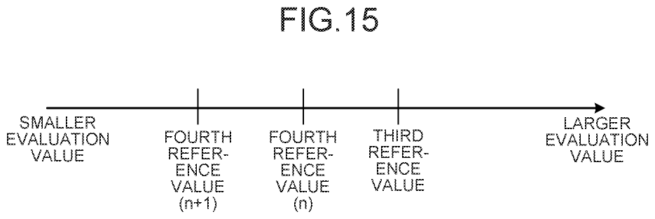

FIG. 15 is a diagram illustrating the specific extraction range.

In this sixth embodiment, the specific extraction range is, as illustrated in FIG. 15, a range having a third reference value and a fourth reference value (n) smaller than the third reference value, the range being less than the third reference value and being less than the fourth reference value (n). The initial value of the fourth reference value is a value that is at least equal to or less than the third reference value.

Specifically, at Step S4A, the control unit 33 determines whether or not the evaluation value is less than the third reference value (Step S41A).

In a case where the control unit 33 has determined that the evaluation value is not less than the third reference value (Step S41A: No), the receiving device 3 proceeds to Step S8.

On the contrary, in a case where the control unit 33 has determined that the evaluation value is less than the third reference value (Step S41A: Yes), the control unit 33 determines whether or not the evaluation value is less than the fourth reference value (Step S42A).

In a case where the control unit 33 has determined that the evaluation value is not less than the fourth reference value (Step S42A: No), the receiving device 3 proceeds to Step S8.

On the contrary, in a case where the control unit 33 has determined that the evaluation value is less than the fourth reference value (Step S42A: Yes), the receiving device 3 proceeds to Step 35.

Step S7A

The control unit 33 updates the specific extraction range by changing the fourth reference value to a smaller value. For example, as illustrated in FIG. 15, the control unit 33 changes the fourth reference value (n) that has been used thus far to a fourth reference value (n+1) smaller than the fourth reference value (n).

Even in a case where the receiving device 3 operates like in the above described sixth embodiment, effects similar to those of the above described first embodiment are thus achieved.

### Seventh Embodiment

A seventh embodiment will be described next.

In the following description, any component that is similar to that of the above described sixth embodiment will be assigned with the same reference sign, and detailed description thereof will be omitted or simplified.

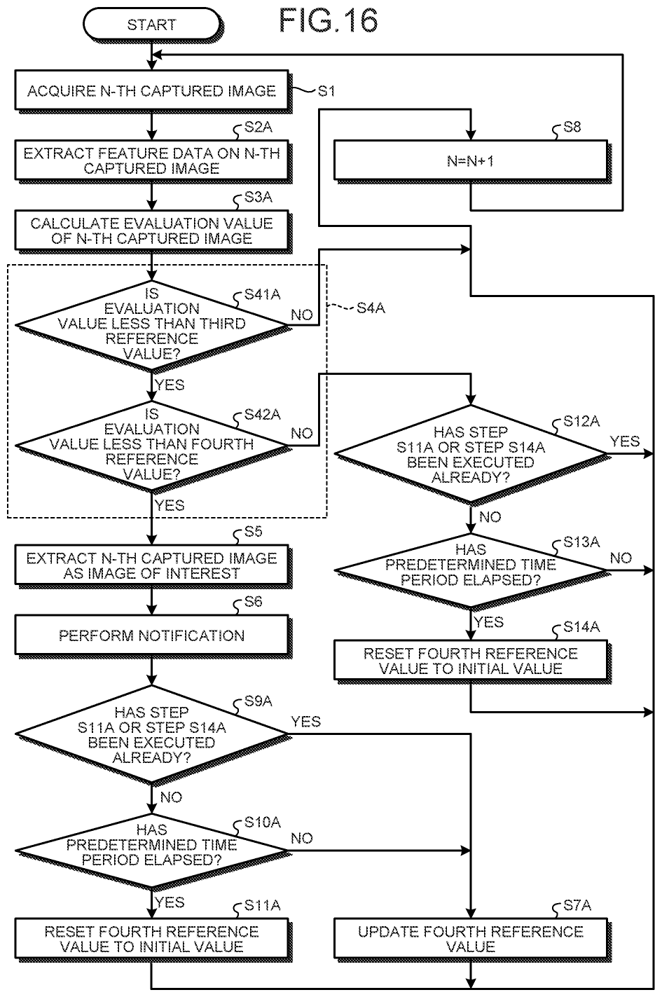

FIG. 16 is a flowchart illustrating operation of a receiving device 3 according to the seventh embodiment.

In this seventh embodiment, as illustrated in FIG. 16, the operation of the receiving device 3 is different from that of the sixth embodiment described above.

Specifically, the seventh embodiment is different from the first embodiment described above in that Steps S9A to S14A have been added in the seventh embodiment. Therefore, Steps S9A to S14A will be described mainly hereinafter.

Step S9A is executed after Step S6.

Specifically, at Step S9A, a control unit 33 determines whether or not resetting of the fourth reference value to the initial value (Step S11A or Step S14A described later) has been executed already.

In a case where the control unit 33 has determined that the resetting of the fourth reference value to the initial value has been executed already (Step S9A: Yes), the receiving device 3 proceeds to Step S7A.

On the contrary, in a case where the control unit 33 has determined that the resetting of the fourth reference value to the initial value has not been executed yet (Step S9A: No), the control unit 33 determines whether or not a predetermined time period has elapsed (Step S10A). For example, at Step S10A, the control unit 33 measures time from a time point at which data on a first image are received and determines whether or not the measured time has become equal to or longer than the predetermined time period.

In a case where the control unit 33 has determined that the predetermined time period has not elapsed (Step S10A: No), the receiving device 3 proceeds to Step S7A.

On the contrary, in a case where the control unit 33 has determined that the predetermined time period has elapsed (Step S10A: Yes), the control unit 33 resets the fourth reference value to the initial value (Step S11A). Thereafter, the receiving device 3 proceeds to Step S8.

Step S12A is executed in a case where the control unit 33 has determined that the evaluation value is not less than the fourth reference value (Step S42A: No).

Specifically, at Step S12A, the control unit 33 determines whether or not the resetting of the fourth reference value to the initial value (Step S11A or later described Step S14A) has been executed already.

In a case where the control unit 33 has determined that the resetting of the fourth reference value to the initial value has been executed already (Step S12A: Yes), the receiving device 3 proceeds to Step S8.

On the contrary, in a case where the control unit 33 has determined that the resetting of the fourth reference value to the initial value has not been executed yet (Step S12A: No), the control unit 33 determines whether or not a predetermined time period has elapsed, similarly to Step S10A (Step S13A).

In a case where the control unit 33 has determined that the predetermined time period has not elapsed (Step S13A: No), the receiving device 3 proceeds to Step S8.

On the contrary, in a case where the control unit 33 has determined that the predetermined time period has elapsed (Step S13A: Yes), the control unit 33 resets the fourth reference value to the initial value (Step S14A). Thereafter, the receiving device 3 proceeds to Step S8.

Even in a case where the receiving device 3 operates like in the above described seventh embodiment, effects similar to those of the above described second and sixth embodiments are thus achieved.

### Eighth Embodiment

An eighth embodiment will be described next.

In the following description, any component that is similar to that of the above described sixth embodiment will be assigned with the same reference sign, and detailed description thereof will be omitted or simplified.

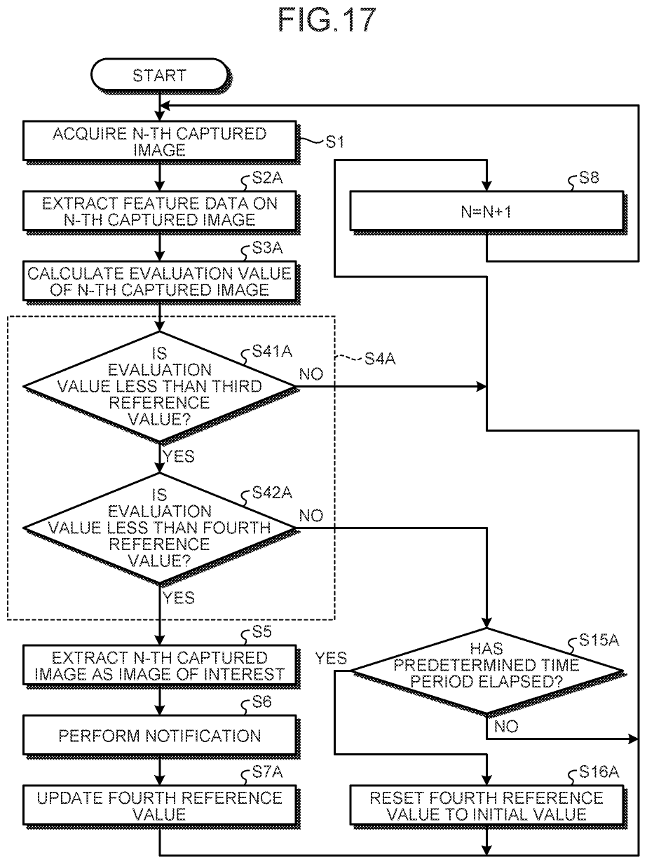

FIG. 17 is a flowchart illustrating operation of a receiving device 3 according to the eighth embodiment.

In this eighth embodiment, as illustrated in FIG. 17, the operation of the receiving device 3 is different from that of the sixth embodiment described above.

Specifically, the eighth embodiment is different from the sixth embodiment described above in that Steps S15A and S16A have been added in the eighth embodiment. Therefore, Steps S15A and S16A will be described mainly hereinafter.

Step S15A is executed in a case where it has been determined that the evaluation value is not less than the fourth reference value (Step S42A: No).

Specifically, at Step S15A, a control unit 33 determines whether or not a predetermined time period has elapsed. For example, the control unit 33 measures a time period over which a state is maintained, the state being where the evaluation value is less than the third reference value but is not less than the fourth reference value, and determines whether or not the time period measured has become equal to or longer the predetermined time period.

In a case where the control unit 33 has determined that the predetermined time period has not elapsed (Step S15A: No), the receiving device 3 proceeds to Step S8.

On the contrary, in a case where the control unit 33 has determined that the predetermined time period has elapsed (Step S15A: Yes), the control unit 33 resets the fourth reference value to the initial value (Step S16A). Thereafter, the receiving device 3 proceeds to Step S8.

Even in a case where the receiving device 3 operates like in the above described seventh embodiment, effects similar to those of the above described third and sixth embodiments are thus achieved.

### Ninth Embodiment

A ninth embodiment will be described next.

In the following description, any component that is similar to that of the above described sixth embodiment will be assigned with the same reference sign, and detailed description thereof will be omitted or simplified.

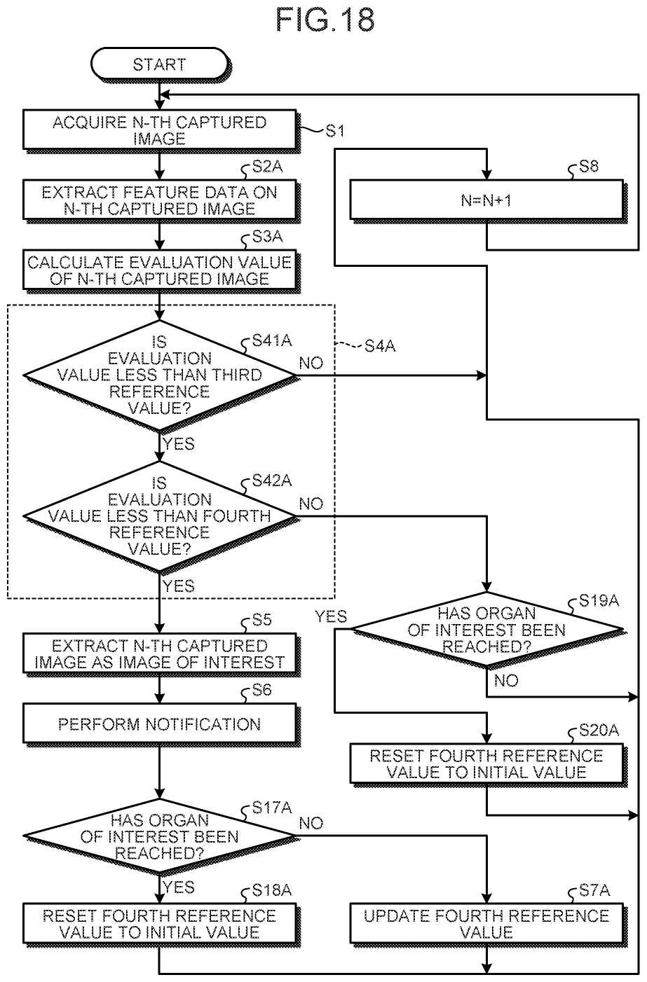

FIG. 18 is a flowchart illustrating operation of a receiving device 3 according to the ninth embodiment.

In this ninth embodiment, as illustrated in FIG. 18, the operation of the receiving device 3 is different from that of the sixth embodiment described above.

Specifically, the ninth embodiment is different from the sixth embodiment described above in that Steps S17A to S20A have been added in the ninth embodiment. Therefore, Steps S17A and S20A will be described mainly hereinafter.

Step S17A is executed after Step S6.

Specifically, at Step S17A, a control unit 33 determines whether or not the capsule endoscope 2 has reached an organ of interest. This organ of interest means at least one specific organ that is an organ present in a path followed by the capsule endoscope 2 and that has been preset. For example, the control unit 33 determines whether or not the capsule endoscope 2 has reached the organ of interest, on the basis of a time period elapsed from a time point at which data on a first image are received, and/or a shape or color of a subject captured in an N-th captured image Pn.

In a case where the control unit 33 has determined that the capsule endoscope 2 has not reached the organ of interest (Step S17A: No), the receiving device 3 proceeds to Step S7A.

On the contrary, in a case where the control unit 33 has determined that the capsule endoscope 2 has reached the organ of interest (Step S17A: Yes), the control unit 33 resets the fourth reference value to the initial value (Step S18A). Thereafter, the receiving device 3 proceeds to Step S8.

Step S19A is executed in a case where it has been determined that the evaluation value is not less than the fourth reference value (Step S42A: No).

Specifically, at Step S19A, the control unit 33 determines whether or not the capsule endoscope 2 has reached the organ of interest, similarly to Step S17A.

In a case where the control unit 33 has determined that the capsule endoscope 2 has not reached the organ of interest (Step S19A: No), the receiving device 3 proceeds to Step S8.

On the contrary, in a case where the control unit 33 has determined that the capsule endoscope 2 has reached the organ of interest (Step S19A: Yes), the control unit 33 resets the fourth reference value to the initial value (Step S20A). Thereafter, the receiving device 3 proceeds to Step S8.

Even in a case where the receiving device 3 operates like in the above described ninth embodiment, effects similar to those of the above described fourth and sixth embodiments are thus achieved.

### Tenth Embodiment

A tenth embodiment will be described next.

In the following description, any component that is similar to that of the above described sixth embodiment will be assigned with the same reference sign, and detailed description thereof will be omitted or simplified.

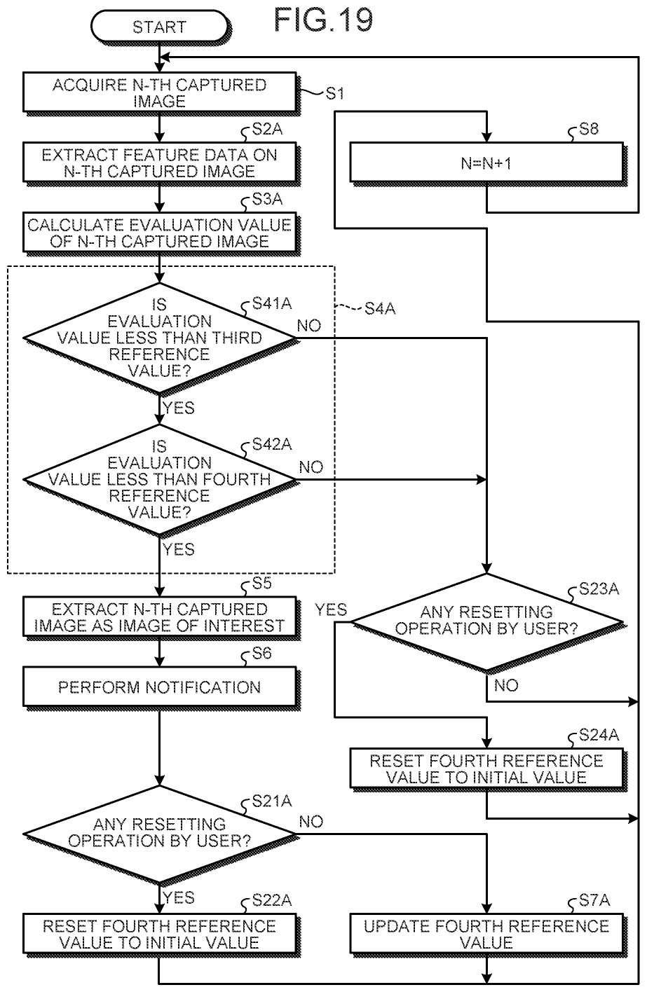

FIG. 19 is a flowchart illustrating operation of a receiving device 3 according to the tenth embodiment.

In the tenth embodiment, as illustrated in FIG. 19, the operation of the receiving device 3 is different from that of the sixth embodiment described above.

Specifically, the tenth embodiment is different from the sixth embodiment described above in that Steps S21A to S24A have been added in the tenth embodiment. Therefore, Steps S21A and S24A will be described mainly hereinafter.

Step S21A is executed after Step S6.

Specifically, at Step S21A, a control unit 33 determines whether or not a resetting operation (user operation) has been made through the operating portion 36 by a user.

Examples of the resetting operation according to this tenth embodiment may include the resetting operation illustrated in FIG. 11 to FIG. 13 in the fifth embodiment described above.

In a case where the control unit 33 has determined that the resetting operation has not been made (Step S21A: No), the receiving device 3 proceeds to Step S7A.

On the contrary, in a case where the control unit 33 has determined that the resetting operation has been made (Step S21A: Yes), the control unit 33 resets the fourth reference value to the initial value (Step S22A). Thereafter, the receiving device 3 proceeds to Step S8.

Step S23A is executed in a case where it has been determined that the evaluation value is not less than the third reference value (Step S41A: No), or in a case where it has been determined that the evaluation value is not less than the fourth reference value (Step S42A: No).

Specifically, at Step S23A, the control unit 33 determines whether or not a resetting operation (user operation) has been made through the operating portion 36 by a user.

In a case where the control unit 33 has determined that the resetting operation has not been made (Step S23A: No), the receiving device 3 proceeds to Step S8.

On the contrary, in a case where the control unit 33 has determined that the resetting operation has been made (Step S23A: Yes), the control unit 33 resets the fourth reference value to the initial value (Step S24A). Thereafter, the receiving device 3 proceeds to Step S8.

Even in a case where the receiving device 3 operates like in the above described tenth embodiment, effects similar to those of the above described fifth and sixth embodiments are thus achieved.

### Other Embodiments

Some embodiments of the disclosure have been described thus far, but the disclosure is not to be limited only to the above described first to tenth embodiments.

In the first to fifth embodiments described above, only one evaluation value of an N-th captured image Pn (the number of pixels having values of R/B, the values exceeding a specific reference value, among all of pixels of the N-th captured image Pn) and the evaluation value is compared with only one first reference value and one second reference value, but the disclosure is not limited to these embodiments. The number of evaluation values calculated may be “n” and these n evaluation values may be compared with n first reference values and n second reference values.

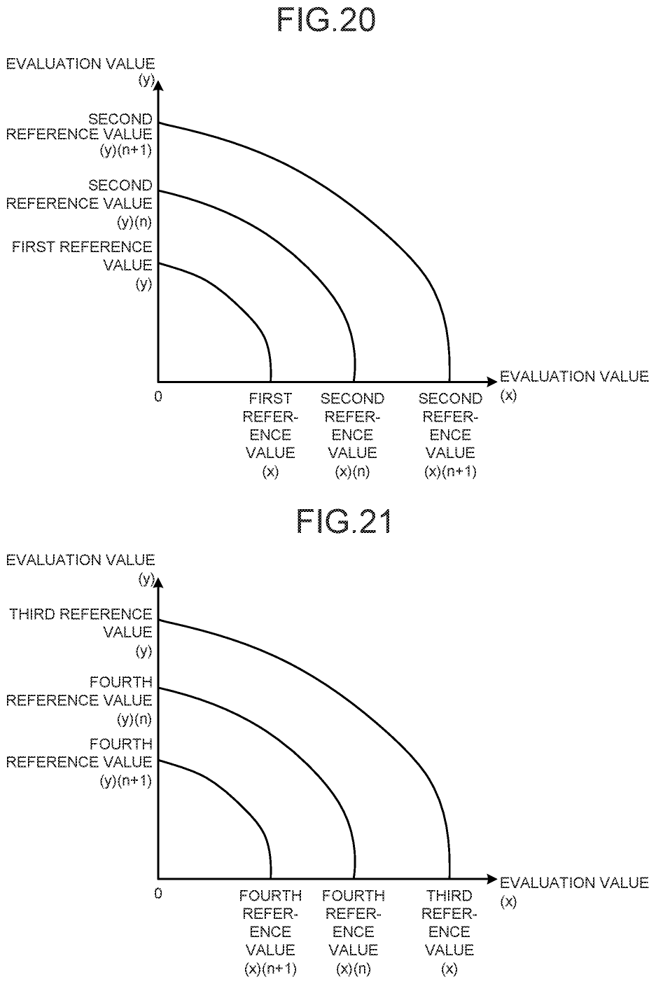

FIG. 20 is a diagram illustrating a modified example of the first to fifth embodiments and exemplifying a case where two evaluation values are calculated and the two evaluation values are compared with two first reference values and two second reference values.

Specifically, a first evaluation value (x) may be, for example, the number of pixels having values of R, the values exceeding a specific reference value, among all of pixels of an N-th captured image Pn. A second evaluation value (y) may be, for example, the largest value of R/B of the pixels of the N-th captured image Pn. Correspondingly to these two evaluation values, the evaluation value (x) and evaluation value (y); two reference values, a first reference value (x) and a first reference value (y), are provided and two reference values, a second reference value (x) (n) and a second reference value (y) (n) are provided. At Step S41, it is determined whether or not the two evaluation values, the evaluation value (x) and evaluation value (y), are outside an area surrounded by an X-axis, a Y-axis, and a line joining the two reference values, the first reference value (x) and first reference value (y). Similarly, at Step S42, it is determined whether or not the two evaluation values, the evaluation value (x) and evaluation value (y), are outside an area surrounded by the X-axis, the Y-axis, and a line joining the two reference values, the second reference value (x) (n) and second reference value (y) (n). Two reference values, a second reference value (x) (n+1) and a second reference value (y) (n+1), illustrated in FIG. 20 are values changed respectively from the two reference values, the second reference value (x) (n) and second reference value (y) (n), at Step S7.

In FIG. 20, the line joining the above described two first reference values and the line joining the above described two second reference values are illustrated as curved lines, but these lines may each be a curved line being part of an ellipse, a curved line being part of a circle, a straight line, or a straight line being part of a rectangle.

In the sixth to tenth embodiments described above, only one evaluation value of an N-th captured image Pn (the smallest value of B/R of pixels of the N-th captured image Pn) is calculated and the evaluation value is compared with only one third reference value and one fourth reference value, but the disclosure is not limited to these embodiments. The number of evaluation values calculated may be “n” and these n evaluation values may be compared with n third reference values and n fourth reference values.

FIG. 21 is a diagram illustrating a modified example of the sixth to tenth embodiments and exemplifying a case where two evaluation values are calculated and the two evaluation values are compared with two third reference values and two fourth reference values.

Specifically, a first evaluation value (x) may be, for example, the smallest value of G/R of pixels of an N-th captured image Pn. A second evaluation value (y) may be, for example, the smallest value of B/R of the pixels of the N-th captured image Pn. Correspondingly to these two evaluation values, the evaluation value (x) and evaluation value (y); two reference values, a third reference value (x) and a third reference value (y), are provided and two reference values, a fourth reference value (x) (n) and a fourth reference value (y) (n), are provided. At Step S41A, it is determined whether or not the two evaluation values, the evaluation value (x) and evaluation value (y), are outside an area surrounded by an X-axis, a Y-axis, and a line joining the two reference values, the third reference value (x) and third reference value (y). Similarly, at Step S42A, it is determined whether or not the two evaluation values, the evaluation value (x) and evaluation value (y), are outside an area surrounded by the X-axis, the Y-axis, and a line joining the two reference values, the fourth reference value (x) (n) and fourth reference value (y) (n). Two reference values, a fourth reference value (x) (n+1) and a fourth reference value (y) (n+1), illustrated in FIG. 21 are values changed respectively from the two reference values, the fourth reference value (x) (n) and fourth reference value (y) (n) at Step S7A.

In FIG. 21, the line joining the above described two third reference values and the line joining the above described two fourth reference values are illustrated as curved lines, but these lines may each be a curved line being part of an ellipse, a curved line being part of a circle, a straight line, or a straight line being part of a rectangle.

In the first to tenth embodiments described above, any of the following evaluation values may be adopted as an evaluation value of an N-th captured image Pn.

For example, in the first to fifth embodiments described above, the largest value of B/R of pixels in an N-th captured image Pn may be adopted as an evaluation value of the N-th captured image Pn.

Furthermore, for example, in the first to tenth embodiments described above, a value resulting from quantification of a lesion or a bleeding site by use of a deep learning technique may be adopted as an evaluation value of an N-th captured image Pn.

In the first to tenth embodiments described above, the receiving device 3 is configured as an image processing device, but without being limited to these embodiments, the image display device 4 may be configured as an image processing device.

In the first to tenth embodiments described above, the configuration to process captured images that have been captured by the capsule endoscope 2 is adopted, but without being limited to this configuration, a configuration to process any other captured images acquired in chronological order may be adopted.

For example, in the first embodiment, the endoscope system 1 including the capsule endoscope 2, the receiving device 3 and the image display device 4 is described, but embodiments are not limited thereto. The endoscope system 1 may include an insertion-type endoscope and the image display device 4. The endoscope may be a medical endoscope or a surgical endoscope. The endoscope may be a flexible endoscope or a rigid endoscope. In the first embodiment, the receiving device 3 connected to the receiving antennas 3a to 3f to be attached to the body surface of the subject 100 is used, but embodiments are not limited thereto. For example, an image processing device that receives image signals transmitted wired or wirelessly from a part of the endoscope that is not inserted into the subject can be used as the receiving device 3.

In addition, flows of the processes are not limited to the sequences of the processes in the flowcharts described above with respect to the first to tenth embodiments, and may be subjected to change so long as no contradiction arises from the change.

An image processing device, an image processing method, and an image processing program, according to the disclosure enable extraction of an image of interest that is a captured image highly needed to be checked by a medical practitioner.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing device, comprising:
a processor comprising hardware, the processor being configured to
calculate an evaluation value of a captured image that is obtained by capturing a subject,
determine whether or not the evaluation value is included in a specific extraction range recorded in a memory,
extract the captured image as an image of interest including a lesion when the processor has determined that the evaluation value is included in the specific extraction range, and
update the specific extraction range based on the evaluation value.

2. The image processing device according to claim 1, further comprising:
an alarm configured to make a notification of a fact that the image of interest has been extracted, or a monitor configured to display the fact, wherein
the processor is configured to perform control to cause the alarm to make the notification of the fact that the image of interest has been extracted or to cause the monitor to display the fact when the captured image has been extracted as the image of interest.

3. The image processing device according to claim 1, wherein the processor is configured to calculate the evaluation value based on feature data on the captured image.

4. The image processing device according to claim 3, wherein the processor is configured to calculate the feature data based on a pixel value of each pixel in the captured image.

5. The image processing device according to claim 1, wherein
the specific extraction range is a range prescribed by a first reference value and a second reference value larger than the first reference value, and
the processor is configured to determine that the evaluation value is included in the specific extraction range when the evaluation value exceeds the first reference value and the evaluation value exceeds the second reference value.

6. The image processing device according to claim 5, wherein the processor is configured to make an update of the specific extraction range such that the evaluation value is the second reference value when the processor has determined that the evaluation value is included in the specific extraction range.

7. The image processing device according to claim 5, wherein the processor is configured to determine whether or not a predetermined time period has elapsed, and when where the processor has determined that the predetermined time period has elapsed, the processor is configured to reset the second reference value to an initial value set before an update.

8. The image processing device according to claim 5, wherein
the captured image is an image captured by an endoscope, and
the processor is configured to determine whether or not the endoscope has reached a specific organ, and when the processor has determined that the endoscope has reached the specific organ, the processor is configured to reset the second reference value to an initial value.

9. The image processing device according to claim 5, further comprising:
an operating portion configured to receive a user operation, wherein
the processor is configured to reset the second reference value to an initial value when the operating portion has received the user operation.

10. The image processing device according to claim 1, wherein
the specific extraction range is a range prescribed by a third reference value and a fourth reference value smaller than the third reference value, and
the processor is configured to determine that the evaluation value is included in the specific extraction range when the evaluation value is less than the third reference value and the evaluation value is less than the fourth reference value.

11. The image processing device according to claim 10, wherein the processor is configured to update the specific extraction range such that the evaluation value is the fourth reference value when the processor has determined that the evaluation value is included in the specific extraction range.

12. The image processing device according to claim 10, wherein the processor is configured to determine whether or not a predetermined time period has elapsed, and when the processor has determined that the predetermined time period has elapsed, the processor is configured to reset the fourth reference value to an initial value.

13. The image processing device according to claim 10, wherein
the captured image is an image captured by an endoscope, and
the processor is configured to determine whether or not the endoscope has reached a specific organ, and in a case where the processor has determined that the endoscope has reached the specific organ, the processor is configured to reset the fourth reference value to an initial value.

14. The image processing device according to claim 10, further comprising:
an operating portion configured to receive a user operation, wherein
the processor is configured to reset the fourth reference value to an initial value when the operating portion has received the user operation.

15. The image processing device according to claim 1,
wherein the evaluation value indicates a degree of importance of the captured image.

16. An image processing method, comprising:
calculating an evaluation value that is an evaluation of a degree of importance of a captured image that is obtained by capturing a subject;
determining whether or not the evaluation value is included in a specific extraction range recorded in a memory;
extracting the captured image as an image of interest including a lesion when it has been determined that the evaluation value is included in the specific extraction range; and
updating the specific extraction range based on the evaluation value.

17. The image processing method according to claim 16, further comprising:
performing control to cause an alarm to make a notification of a fact that the image of interest has been extracted or to cause a monitor to display the fact when the captured image has been extracted as the image of interest.

18. The image processing method according to claim 16, further comprising,
calculating the evaluation value based on feature data on the captured image.

19. The image processing method according to claim 18, further comprising
calculating the feature data based on a pixel value of each pixel in the captured image.

20. A non-transitory computer-readable recording medium with an executable program stored thereon, the program causing a processor to execute:
calculating an evaluation value that is an evaluation of a degree of importance of a captured image that is obtained by capturing a subject;
determining whether or not the evaluation value is included in a specific extraction range recorded in a memory;
extracting the captured image as an image of interest including a lesion when it has been determined that the evaluation value is included in the specific extraction range; and
updating the specific extraction range based on the evaluation value.
